(19) United States
(12) Patent Application Publication
(10) Pub. No.: US 2006/0241664 A1
(43) Pub. Date: Oct. 26, 2006

(54) Automatic retractable blade scalpel

(75) Inventor: Choi Fat Lam, Houston, TX (US)

Correspondence Address: Richard L. Moseley, P.O. Box 630708, Houston, TX, 77263, US

(73) Assignee: GLOBE MEDICAL TECH, INC.

(21) Appl. No.: 11/115,446

(22) Filed: Apr. 25, 2005

(51) Int. Cl. A61B 17/32

(52) U.S. Cl. 606/167

(57) ABSTRACT

There is provided a disposable scalpel having an automatically retractable blade. The blade may be secured in any of three positions: 1) an intermediated retracted position for shipping, 2) an exposed position for use and 3) a permanently locked retracted position. A spring is provided which automatically retracts the blade to the desire position.

### BACKGROUND OF THE INVENTION

1. Field of the Invention

The present invention relates generally to surgical knives and more particularly to disposable scalpels. More particularly the invention relates to a disposable scalpel which may be retracted into the handle for safe disposal. Most particularly the invention relates to a disposable scalpel which is automatically retracted into the handle and is locked in the final retracted position to prevent further use.

2. Related Art

Surgical scalpels are special knives, the blades of which are manufactured to precise standards of high quality materials. For various reasons, it has become common to use a scalpel blade only once. Therefore there are several combination handle/replaceable blade scalpels disclosed in the art. Additionally there are now available completely disposable scalpels having inexpensive plastic handles secured to the scalpel blade.

With the advent of the AIDS virus, there has now arisen much concern about transmission of disease by contact with contaminated blood. Sharp instruments, such as scalpels, which routinely come into contact with body fluids, are particularly worrisome. For this reason scalpels have been provided with removable guards to prevent contact with the blade when not in use. See for example U.S. Pat. No. 4,735,202 which discloses a scalpel in which the glade guard is provided in the form of a sleeve which is slidably mounted over the scalpel and may be locked into position over the blade when not in use. The blade guard requires two hands to position. Additionally U.S. Pat. No. 5,139,507 discloses such a scalpel whose guard may be easily moved into or out of position by the surgeon during actual surgery to prevent accidental cutting or jabbing when passing the instrument back and forth during surgery.

After any “disposable” scalpel or blade is used there is still the problem of disposing of it. The scalpels or blades must be placed in special “sharps” containers to prevent janitorial or other clean up personnel from coming into contact with the contaminated instruments. Even if the blade is covered by a guard or sheath, there is a chance that the guard might become retracted or removed. Additionally, there is always the temptation to reuse such instruments, especially in poorer areas where disposal of a “perfectly good scalpel” might appear as a waste.

A disposable scalpel with a retractable blade which is locked in the final retractable position is disclosed in U.S. Pat. No. 5,330,493. A spring loaded automatically retractable scalpel is disclosed in U.S. Pat. No. 5,403,337.

Broadly it is an object of the present invention to provide a scalpel with a retractable blade such that the blade can be positioned for use and retracted into the handle after use.

It is another object of the invention to provide a retractable blade scalpel that when fully retracted is permanently locked in the retractable position to prevent accidental reopening or reuse.

It is a further object of the invention to provide a scalpel whose blade is automatically retracted into the handle.

### SUMMARY OF THE INVENTION

To achieve the above objects a scalpel is provided with a blade that is retractable into the handle. The blade may be positioned in three locations within the handle: 1) retracted for shipment, 2) exposed for use and 3) fully retracted and locked for disposal. The blade is automatically retractable into the handle by a spring which is in tension when the blade is in the first two positions and which pull the blade from the exposed to the other two positions. A means is provided to prevent the blade being retracted into the final locked position unless desired.

More particularly the retractable blade scalpel comprises

(a) a handle defining a chamber at the distal end;

(b) a surgical blade slidably mounted within said chamber;

(c) first means secured to said blade and extending externally of said chamber to move said blade from a first exposed position to a second intermediate position in said chamber and to a third position completely within said chamber;

(d) second means to releasably lock said blade in said exposed position;

(e) third means to releasably lock said blade in said intermediate position;

(f) fourth means to permanently lock said blade in said third position; and

(g) a spring secured to said blade and the inside of said handle to automatically move said blade to each of said positions when said first means is activated.

### BRIEF DESCRIPTION OF THE DRAWING

FIG. 1 is an exploded view of the preferred embodiment of the invention.

FIG. 2 is an exploded view of the embodiment of FIG. 1 shown from the opposite angle.

FIG. 3 is a side view of the preferred embodiment with the cover removed and the blade in the temporary locked position as for shipping.

FIG. 4 is a side view of the preferred embodiment with the cover removed and the blade in the exposed position.

FIG. 5 is a side elevational view of the preferred embodiment with case removed and the blade in the permanent locked position.

FIG. 6 is a view along 6-6 of FIG. 5 in partial cross section.

### DESCRIPTION OF THE PREFERRED EMBODIMENT

For a detailed description of the preferred embodiment the reader is directed to the accompanying figures in which like components are given like numerals for ease of reference.

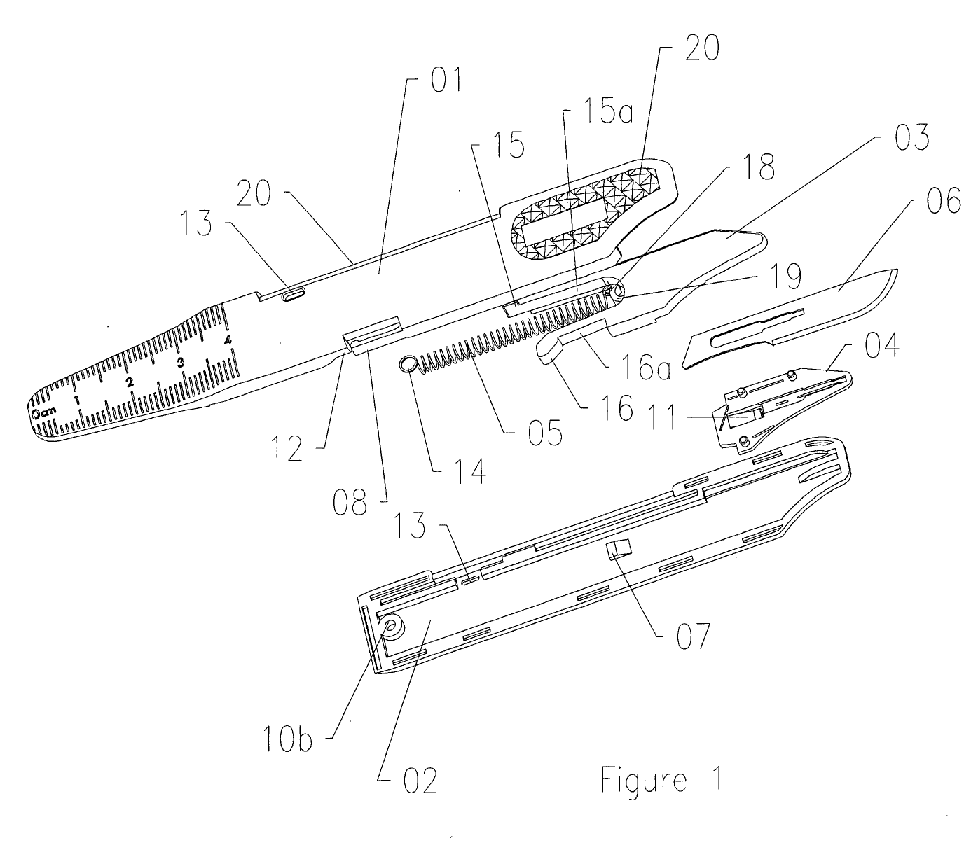

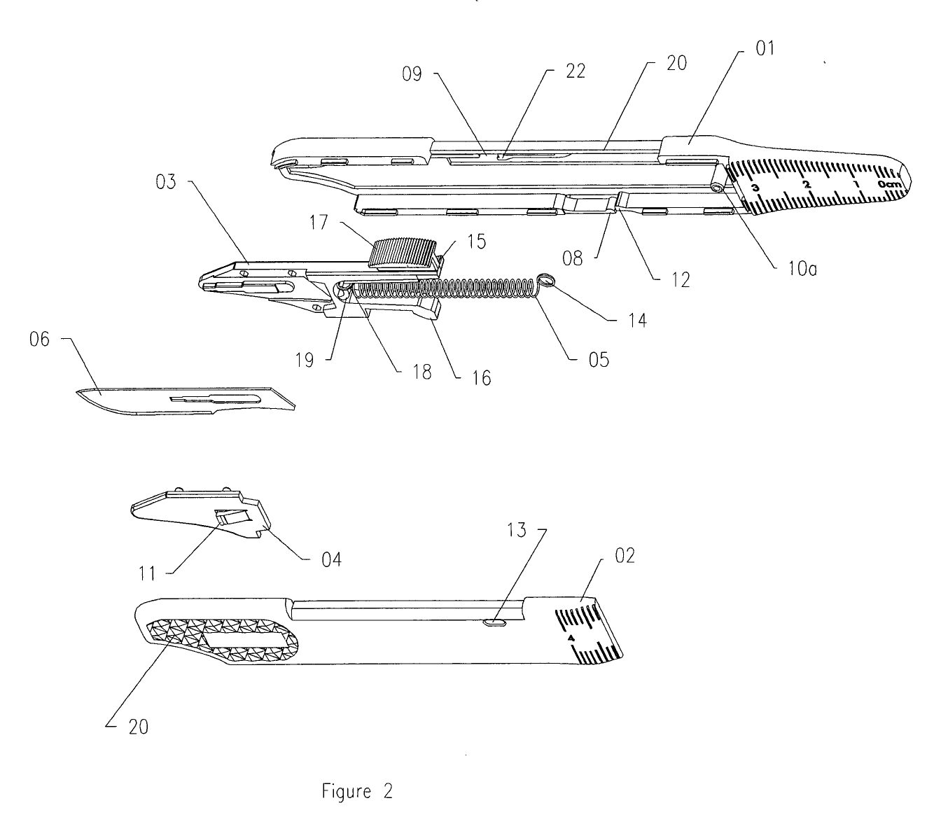

Referring first to FIGS. 1 and 2 there is shown exploded views of the preferred embodiment. The scalpel is seen to comprise a case 01 having a knurled surface 20 at the proximal end and a centimeter scale at the distal end. The knurled surface 20 is preferably an over mold of softer material to provide a better grip. On the lower edge of case 01 is a flexible tab 08 which is opposite temporary locking ramp surface 12.

A surgical blade 06 is mounted onto a blade carrier 03 by mounting plate 04. On one side surface of the carrier 03 is permanent locking tab 11. On the lower edge of the blade carrier 03 is a flexible member 16a at the end of which is a temporary locking tab 16. Exposure locking tab 15 on flexible member 15a engages an exposure locking surface 22 on the interior surface of the case 01 to hold the blade 06 and carrier 03 in the exposed position. Exposure locking tab 15 extend through slot 20 for access. Flexible tab 08 on the lower edge of case 01 is in contact with temporary locking tab 16. A spring 05 is secured to blade carrier by loop 18 which engages hook 19. The loop 14 at the distal end of the spring is secured by pin 10a which engages pin receptacle 10b on cover 02. On the inside surface of cover 02 there is permanent locking ramp 07 which is engaged by permanent locking tab 11 when the blade is secured in the permanently locked position (see below).

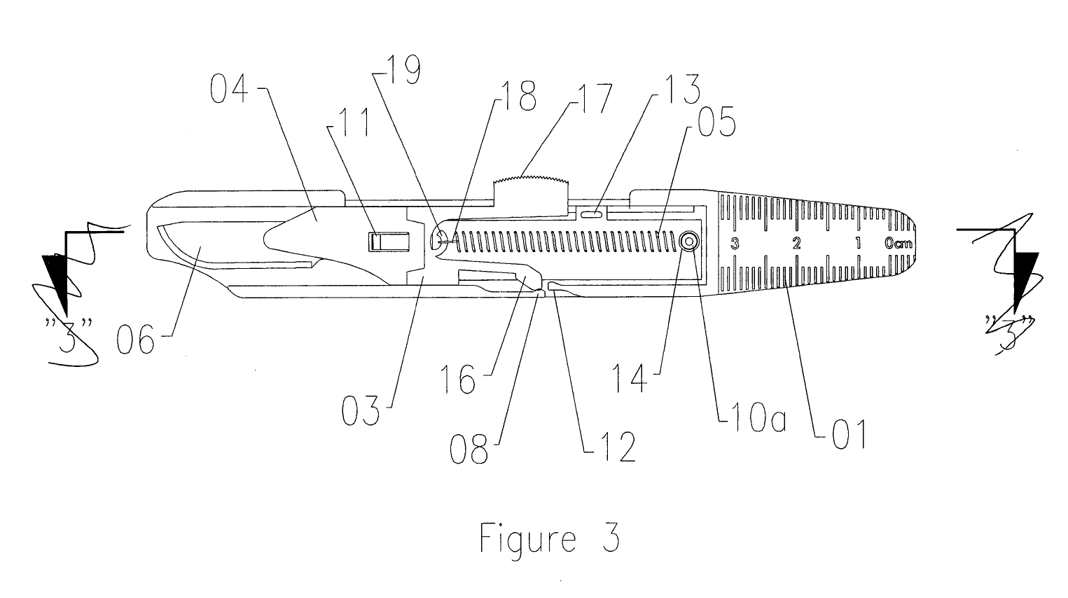

Referring now to FIG. 3 a side elevational view of the scalpel with the cover removed is shown. This configuration has the blade retracted and releasably locked in the intermediate position as for shipping or for when the scalpel is to be passed from one person to another. In this position the temporary locking tab 16 is engaged against flexible tab 08 which prevents further retraction of the blade within the case. The spring 05 is stretched in a tension mode in this position.

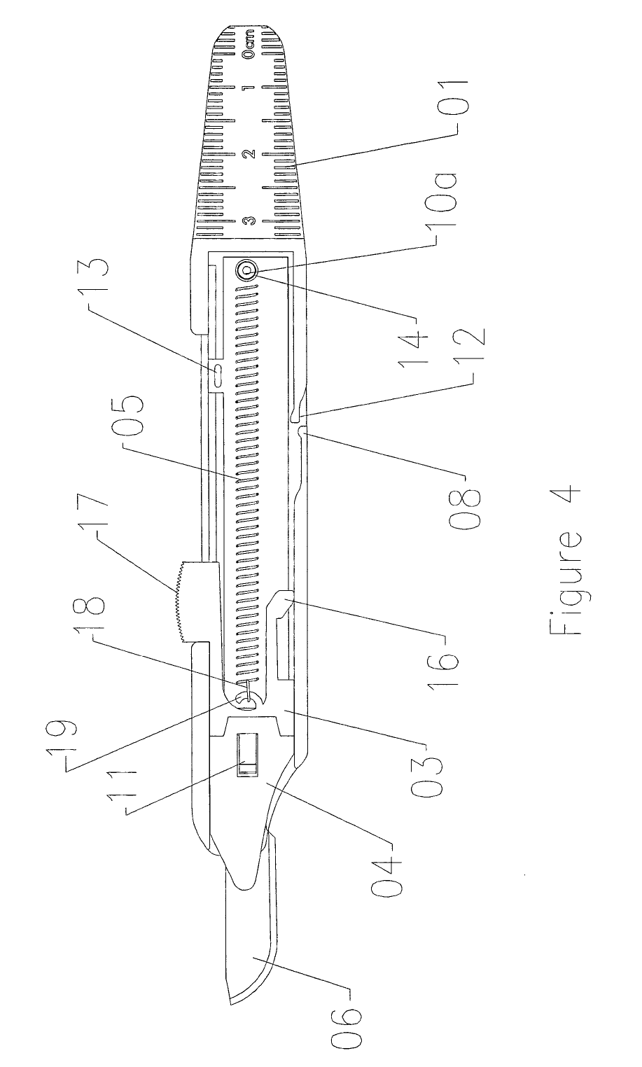

Referring now to FIG. 4 a side elevational view of the scalpel with cover removed. This configuration show the blade in the exposed position. Exposure locking tab 15 has engaged exposure locking surface 22 to hold the blade and carrier in the exposed position. The spring 05 is stretched to a further tension mode.

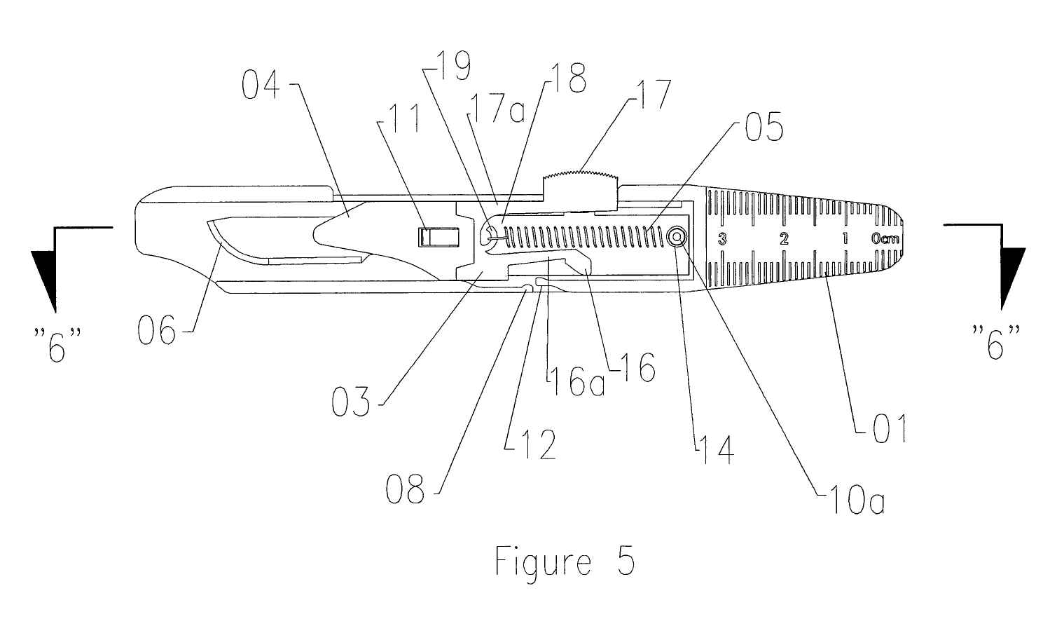

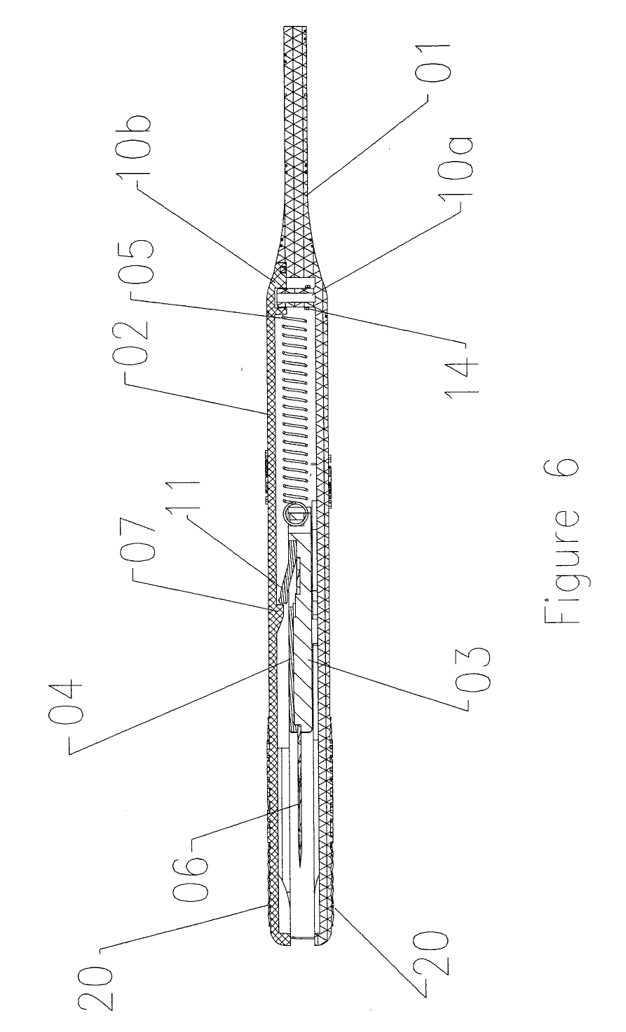

Finally the blade and carrier are shown in the permanent locked position in FIGS. 5 and 6. Permanent locking tab 11 on mounting plate 04 has engaged permanent locking ramp 11 on the interior surface of the case. The tension of spring 05 has pulled the carrier 03 and blade 06 backed to this position.

In use the scalpel is shipped with the blade in the intermediate retracted position with the blade fully within the case. The user presses down on the exposure locking tab 15 to move the blade forward into the exposed position and extend the spring. The exposure locking tab engages exposure locking surface 22 to hold the blade in the exposed position. If the blade needs to be temporarily retracted the user presses down on the exposure locking tab 15 which releases the carrier and allows the spring 05 in tension to retract the blade to the intermediate position. Temporary locking tab 16 engages the temporary locking ramp surface 12 to prevent the blade from being retracted further.

After the scalpel has been used and is ready to be discarded the blade is permanently locked by pressing inward on flexible member 08 which in turn presses inwardly against flexible member 16a which releases the temporary locking tab 16 from locking ramp surface 12. The spring 05, still in tension, automatically retracts the blade to the permanently locked position. The user can view the carrier through viewing holes 13 on case and cover to note that it is in the locked position. The carrier cannot be disengaged from the permanent locking ramp without removing the cover and taking the scalpel apart.

The invention claimed is:

1. A surgical scalpel comprising:
(a) a handle having a proximate end and a distal end and defining a chamber;
(b) a surgical blade slidably mounted within said chamber;
(c) first means secured to said blade and extending externally of said chamber to move said blade from a first exposed position to a second intermediate position in said chamber and to a third position completely within said chamber;
(d) second means to releasably lock said blade in said exposed position;
(e) third means to releasably lock said blade in said intermediate position;
(f) fourth means to permanently lock said blade in said third position; and
(g) a spring secured to said blade and the inside of said handle to automatically move said blade to each of said positions when said first means is activated.

2. The retractable blade scalpel according to claim 1 wherein said blade is mounted on a blade carrier which is slidably mounted within said handle, said blade carrier having an upper surface, a lower surface and two side surfaces, and said first means comprises a first tab connected to the upper surface of said blade carrier by a flexible member which is biased upward.

3. The retractable blade scalpel according to claim 2 wherein said second means comprises a exposure locking surface extending from the interior surface of said chamber and an exposure locking tab extending from said first tab to engage said exposure locking surface.

4. The retractable blade scalpel according to claim 2 wherein said third means comprises a second flexible member extending downward from the lower surface of said blade carrier which engages a temporary locking tab entending upward from the lower interior surface of said chamber.

5. The retractable blade scalpel according to claim 2 wherein said fourth means comprises a permanent locking ramp on the internal surface of said chamber and a permanent locking tab extending from the side surface of said blade carrier.

6. The retractable blade scalpel according to claim 1 wherein said spring is in tension when said blade carrier is in the exposed and temporary locked position.

7. The retractable blade scalpel according to claim 1 further comprising a soft knurled surface over molded on said proximal end.

8. A retractable blade scalpel comprising:
(a) a handle having a proximal end and a distal end and defining a chamber having an open slot at the upper end and having a permanent locking ramp on the internal surface of said chamber;
(b) a first flexible member on the lower on the lower edge of said handle;
(c) a temporary locking ramp surface on the lower edge of said handle opposite said first flexible member;
(d) a surgical blade mounted on a blade carrier which is slidably secured within said chamber;
(e) a permanent locking tab on the side of said blade carrier which can engage said permanent locking ramp;
(f) an exposure locking tab connected to said blade carrier by a second flexible member;
(g) an exposure locking surface on the interior of said chamber which can engage said exposure locking tab;
(h) a temporary locking tab connected to said blade carrier by a third flexible member, said temporary locking tab biased downward and into engagement with said temporary locking ramp surface by said third flexible member, said temporary locking tab being in contact with said first flexible member such that said temporary locking tab is disengaged from said temporary locking ramp surface by inward pressure on said first flexible member; and
(i) a soft knurled surface over molded on said proximal end.
